(19) United States
(12) Patent Application Publication
(10) Pub. No.: US 2026/0266293 A1
(43) Pub. Date: Sep. 10, 2026

(54) BLOOD PUMP

(71) Applicant: ABIOMED Europe GmbH, Aachen (DE)

(72) Inventors: Frank Kirchhoff, Aachen (DE); Werner Carell, Aachen (DE)

(21) Appl. No.: 19/491,433

(22) PCT Filed: Jun. 13, 2024
(86) PCT No.: PCT/EP2024/066473

§ 371 Date: Dec. 9, 2025

(30) Foreign Application Priority Data
Jun. 27, 2023 (EP) 23181888.1

(51) Int. Cl. H05K 1/11; A61M 60/13; A61M 60/216; A61M 60/414; F04D 7/00; F04D 13/06; H02K 7/14; H02K 11/00; H05K 1/16

(52) U.S. Cl. CPC H05K 1/111; A61M 60/13; A61M 60/216; A61M 60/414; F04D 7/00; F04D 13/06; H02K 7/14; H02K 11/0094; H05K 1/165; H05K 2201/10356

(57) ABSTRACT

The present disclosure relates to a blood pump (10) comprising a pumping section (12) having a first blood flow opening (14) and a second blood flow opening (16), a pump element (18) being at least partially disposed within the pumping section (12), a drive unit (20) having a rotor (22) and a stator (24), wherein the stator (24) comprises a first coil assembly (26) and second coil assembly (28), and a circuit board (32) having a first pad of a first type (P11), a second pad of the first type (P21), a first pad of a second type (P12) and a second pad of the second type (P22). The first pad of the second type (P12) and the second pad of the second type (P22) are distanced from each other on the circuit board (32) and the first pad of the second type (P12) is electrically connected to the second pad of the second type (P22) by a first bridge (34). The first coil assembly (26) is connected to the first pad of the first type (P11) and to the first pad of the second type (P12), and the second coil assembly (28) is connected to the second pad of the first type (P21) and to the second pad of the second type (P22). A first cable (42) is attached to the first pad of the first type (P11) and a second cable (44) is attached to the second pad of the first type (P21). The first cable (42) and the second cable (44) are configured to apply electrical signals to the first coil assembly (26) and the second coil assembly (28) to rotate the rotor (22) and to drive the pump element (18) so that a blood flow between the first blood flow opening (14) and the second blood flow opening (16) is generated.

The present invention relates to a blood pump.

### BACKGROUND OF THE INVENTION

Different types of blood pumps are known, such as axial blood pumps, centrifugal (i.e. radial) blood pumps or combinations of both possibilities, where the blood flow is caused by both radial and axial forces. Such blood pumps are intended to support a patient's heart and are generally inserted into the patient's heart via a blood vessel such as the aorta or femoral artery by means of a catheter through a vascular access in the patient's skin, i.e. percutaneously.

Such blood pumps typically comprise a pumping section having a first blood flow opening and a second blood flow opening. The pump element may be an impeller having blades on its outer circumference.

The pump element is driven by a drive unit and causes a blood flow between the at least one first blood flow opening and the at least one second blood flow opening. The blood pump may be provided for left ventricular support so that a blood flow is generated from the first blood flow opening to the second blood flow opening. In this case, the first blood flow opening is a blood flow inlet and the second blood flow opening is a blood flow outlet. Such a blood pump is also called an intravascular blood pump. Further, blood pumps which are placed in a patient's heart may also be referred to as intracardiac blood pumps. Of course, such blood pumps may also be used as a right ventricular support and the blood pumps do not necessarily need to be deployed within the patient's heart or blood vessels. Rather, such blood pumps may also be deployed externally of the heart to establish a blood flow from the heart to the blood vessel or vice versa.

To establish the blood flow, the pump element is at least partially disposed within the pumping section and is generally supported on a shaft or axle, which is driven by the drive unit so that the pump element rotates at a chosen speed to generate the blood flow. Therefore, the drive unit typically comprises a rotor and a stator. The stator may comprise several coil assemblies which are connected to a circuit board. Electrical signals are transmitted to the coil assemblies by cables attached to the circuit board, so that a rotating magnetic field is established by the coil assemblies which causes the rotor to rotate and to thus drive the pump element.

Typically, the circuit board comprises a plurality of pads to which the cables and the coil assemblies are mutually connected by forming nodes e.g., by brazing or soldering. However, as the circuit board has a very small dimension and a plurality of cables and coils are to be attached to the circuit board, the connecting process is demanding and may not be automatized, as typically two or more cables are to be soldered to one pad. In addition, the cables and connection wires of the coil assemblies need to be guided to the respective pad e.g., along the circumference of the circuit board, which may be demanding as bypass currents are to be avoided.

Therefore, some of the connections or nodes respectively are formed via pads provided on an opposite surface of the circuit board. Typically, the connection of the coil assemblies is in a so-called star or wye circuit configuration, with the respective central node being formed via a larger pad provided on the opposite surface of the circuit board. This has the advantage that this space available on both sides of the circuit board can be utilized. However, the production of such a circuit board is cost intensive and may only be automatized to a certain degree. Alternatively, the central node may be formed by twisting and soldering the appropriate wires together, as derivable from e.g., WO 2023/018793 A1. However, this configuration utilizes a lot of space and it needs to be warranted that no bypass currents emerge or unwanted contact between the wires and the pads or another wire is unintentionally established.

It is thus an object of the invention to provide a blood pump having an improved circuit board, which allows for simplified assembly.

### SUMMARY OF THE INVENTION

According to a first aspect, the blood pump may comprise a pumping section, a pump element, a drive unit, a circuit board, a first cable and a second cable. The pumping section may be a pump housing.

The pumping section may have a first blood flow opening and a second blood flow opening. The pump element may at least partially be disposed within the pumping section. The drive unit may have a rotor and a stator, and the stator may comprise a first coil assembly and second coil assembly. The circuit board may have a first pad of a first type, a second pad of the first type, a first pad of a second type and a second pad of the second type. The first pad of the second type and the second pad of the second type may be distanced from each other on the circuit board. In other words, the first pad of the second type and the second pad of the second type may be spatially separated from each other on the circuit board The first pad of the second type may be electrically connected to the second pad of the second type by a first bridge. The first coil assembly may be connected to the first pad of the first type and to the first pad of the second type. The second coil assembly may be connected to the second pad of the first type and to the second pad of the second type. The first cable may be attached to the first pad of the first type. The second cable may be attached to the second pad of the first type. The first cable and the second cable may be configured to apply electrical signals to the first coil assembly and the second coil assembly to rotate the rotor and to drive the pump element so that a blood flow between the first blood flow opening and the second blood flow opening is generated.

Due to the distance between the first pad of the second type and the second pad of the second type, the coil assemblies may easily be connected to one another to form the intended circuit configuration. The electrical connection between the first pad of the second type and second pad of the second type is established via the bridge, so that not one large pad is provided, but rather two smaller pads which can be optimally provided on the circuit board for connection to the respective coil assembly.

Accordingly, the two pads can be provided at a desired distance from each other, whilst still being electrically connected by the bridge. The bridge only slightly increases the total thickness of the circuit board which does not or only insignificantly affect the overall length of the blood pump.

Preferably, the first coil assembly comprises a first coil of a first type and a second coil of the first type. The circuit board may comprise a first pad of a third type and a second pad of the third type, wherein the first pad of the third type and the second pad of the third type are distanced from each other on the circuit board. In other words, the first pad of the third type and the second pad of the third type may be spatially distanced from each other on the circuit board The first pad of the third type may be electrically connected to the second pad of the third type by a second bridge. The first coil of the first type may be connected to the first pad of the first type to establish the connection between the first coil assembly and the first pad of the first type. The second coil of the first type may be connected to the first pad of the second type to establish the connection between the first coil assembly and the first pad of the second type. The first coil of the first type may be connected to the first pad of the third type, and the second coil of the first type may be connected to the second pad of the third type.

Accordingly, the first coil assembly may be of the multi-layer type. The first coil of the first type and the second coil of the first type may thus be provided layered. Alternatively, the first coil of the first type and the second coil of the first type may be provided opposite to each other in a two-slot configuration.

Preferably, the second coil assembly comprises a first coil of a second type and a second coil of the second type. The circuit board may comprise a first pad of a fourth type and a second pad of the fourth type. The first pad of the fourth type and the second pad of the fourth type may be distanced from each other on the circuit board. In other words, the first pad of the fourth type and the second pad of the fourth type may be spatially separated from each other on the circuit board. The first pad of the fourth type may be electrically connected to the second pad of the fourth type by a third bridge. The first coil of the second type may be connected to the second pad of the first type to establish the connection between the second coil assembly and the second pad of the first type. The second coil of the second type may be connected to the second pad of the second type to establish the connection between the second coil assembly and the second pad of the second type. The first coil of the second type may be connected to the first pad of the fourth type and the second coil of the second type may be connected to the second pad of the fourth type.

Accordingly, the second coil assembly may be of the multi-layer type. The first coil of the second type and the second coil of the second type may thus be provided layered. Alternatively, the first coil of the second type and the second coil of the second type may be provided opposite to each other in a two-slot configuration.

Preferably, the stator comprises a third coil assembly. The circuit board may comprise a third pad of the first type and a third pad of the second type. The first pad of the second type and the second pad of the second type and the third pad of the second type may be distanced from each other on the circuit board. In other words, the first pad of the second type and the second pad of the second type and the third pad of the second type may be spatially separated from each other on the circuit board The third pad of the second type may be electrically connected to the first pad of the second type and the second pad of the second type by the first bridge. The third coil assembly may be connected to the third pad of the first type and to the third pad of the second type. The blood pump may further comprise a third cable being attached to the third pad of the first type. The third cable may be configured to apply electrical signals to the third coil assembly.

The electrical signals establish the rotating magnetic field as to rotate the rotor and to drive the pump element so that the blood flow between the first blood flow opening and the second blood flow opening is generated.

Thus, the drive unit may be a three-phase electric motor. In particular, the drive unit may be a three-phase two-pole electric motor.

Preferably, the third coil assembly comprises a first coil of a third type and a second coil of the third type. The circuit board may comprise a first pad of a fifth type and a second pad of the fifth type. The first pad of the fifth type and the second pad of the fifth type may be distanced from each other on the circuit board. In other words, the first pad of the fifth type and the second pad of the fifth type may be distanced from each other on the circuit board The first pad of the fifth type may be electrically connected to the second pad of the fifth type by a fourth bridge. The first coil of the third type may be connected to the third pad of the first type to establish the connection between the third coil assembly and the third pad of the first type. The second coil of the third type may be connected to the third pad of the second type to establish the connection between the third coil assembly and the third pad of the second type. The first coil of the third type may be connected to the first pad of the fifth type. The second coil of the third type may be connected to the second pad of the fifth type.

Accordingly, the third coil assembly may be of the multi-layer type. The first coil of the third type and the second coil of the third type may thus be provided layered. Alternatively, the first coil of the third type and the second coil of the third type may be provided opposite to each other in a two-slot configuration.

The circuit board may comprise a first layer having a first side and a second side. The first pad of the first type and the second pad of the first type and the first pad of the second type and the second pad of the second type may be disposed on the first side of the first layer. The first bridge may be disposed on the second side of the first layer. Placing the bridge on the second side of the first layer saves space on the first side of the first layer for the pads.

The first layer may comprise a first opening and a second opening. The circuit board may comprise a first connector and a second connector. The first connector may extend through the first opening. The second connector may extend through the second opening. The first bridge may be connected to the first connector and to the second connector. The first pad of the second type may be connected to the first connector and the second pad of the second type may be connected to the second connector.

The connectors allow for an electrical connection between the bridge and the first pad of the second type and the second pad of the first type.

The third pad of the first type and the third pad of the second type may be disposed on the first side of the first layer. The first layer may comprise a third opening and the circuit board may comprise a third connector that may extend through the third opening. The first bridge may be connected to the third connector and the third pad of the second type may be connected to the third connector. The third connector allows for an electrical connection between the first bridge and the third pad of the second type.

Preferably, the circuit board comprises a second layer having a first side and a second side. The first bridge may be disposed on the first side of the second layer. Thus, the circuit board may have a multi-layer configuration.

The first pad of the third type and the second pad of the third type may be disposed on the first side of the first layer. The first pad of the fourth type and the second pad of the fourth type may be disposed on the first side of the first layer. The first pad of the fifth type and the second pad of the fifth type may be disposed on the first side of the first layer. Thus, the pads intended for connection to the coils may all be disposed on the first side of the first layer.

Preferably, the second bridge is disposed on the second side of the second layer. The first layer may comprise a fourth opening and a fifth opening. The second layer may comprise a sixth opening and a seventh opening. The circuit board may comprise a fourth connector and a fifth connector. The fourth connector may extend through the fourth opening and the sixth opening and the fifth connector may extend through the fifth opening and the seventh opening. The second bridge may be connected to the fourth connector and the fifth connector. The first pad of the third type may be connected to the fourth connector and the second pad of the third type may be connected to the fifth connector.

Preferably, the third bridge is disposed on the second side of the second layer. The first layer may comprise an eighth opening and a ninth opening and the second layer may comprise a tenth opening and an eleventh opening. The circuit board may comprise a sixth connector and a seventh connector. The sixth connector may extend through the eighth opening and the tenth opening and the seventh connector may extend through the ninth opening and the eleventh opening. The third bridge may be connected to the sixth connector and the seventh connector. The first pad of the fourth type may be connected to the sixth connector and the second pad of the fourth type may be connected to the seventh connector.

Preferably, the fourth bridge is disposed on the second side of the second layer. The first layer may comprise a twelfth opening and a thirteenth opening and the second layer may comprise a fourteenth opening and a fifteenth opening. The circuit board may comprise an eighth connector and a ninth connector. The eighth connector may extend through the twelfth opening and the fourteenth opening and the ninth connector may extend through the thirteenth opening and the fifteenth opening. The fourth bridge may be connected to the eighth connector and the ninth connector. The first pad of the fifth type may be connected to the eighth connector and the second pad of the fifth type may be connected to the ninth connector.

Placing at least one of the second to third bridge on the second side of the second layer of the circuit board saves space on the first side of the first layer and further allows for flexible placement of the respective pads on the first side of the first layer, as the electrical connection between the pads is established via the respective connectors and the respective bridge.

The circuit board may comprise an adhesive foil on the opposite side of the first side of the first layer. Preferably, the adhesive foil is provided on the second side of the second layer. The circuit board can thus easily be attached to the respective component of the blood pump e.g., the stator.

The pads may be disposed in an annular pattern or ring shaped pattern respectively on the circuit board, wherein in between two pads of the same type at least one pad of a different type is located.

Preferably, in between all pads of the same type at least one pad of a different type is located. This allows for flexible attachment of the cables and coil assemblies to the circuit board and greatly reduces the need of guiding cables and wires along the outer circumference of the circuit board or the stator respectively.

Preferably, the circuit board has an annular shape. The circuit board may have a central opening. The circuit board may have an outer diameter of 2 mm to 4 mm, preferably of 3 mm. The central opening may have a diameter of 1 mm to 2 mm, preferably of 1.6 mm. The circuit board may have a thickness in the axial direction of 0.1 mm to 0.3 mm, preferably of 0.22 mm.

The first layer may have a thickness in the axial direction of 0.02 mm to 0.07 mm, preferably of 0.05 mm. The first layer may comprise a polyimide or may be composed of a polyimide. The second layer may have a thickness in the axial direction of 0.02 mm to 0.07 mm, preferably of 0.05 mm. The second layer may comprise a polyimide or may be composed of a polyimide.

At least one or even all of the pads may comprise copper or may be made of copper. At least one or even all of the pads may be gold plated. At least one or even all of the pads may have a thickness in the axial direction of 0.015 mm to 0.055 mm, preferably of 0.035 mm.

At least one or even all of the bridges may comprise copper or may be made of copper. At least one or even all of the bridges may be gold plated. At least one or even all of the bridges may have a thickness in the axial direction of 0.015 mm to 0.055 mm, preferably of 0.035 mm.

The adhesive foil may have a thickness in the axial direction of 0.02 mm to 0.07 mm, preferably of 0.05 mm.

At least one or even all of the openings may have diameter of 0.05 mm to 0.15 mm, preferably of 0.1 mm.

At least one or even all of the connectors may comprise copper or may be made of copper. At least one or even all of the connectors may be gold plated. One or all connectors may be bolt-shaped. At least one or even all connectors may have a diameter 0.08 mm to 0.12 mm, preferably of 0.1 mm.

The terms “distanced” or “spatially separated” as used herein are to be understood in that the respective pads are provided at a distance from each other on the circuit board, so that there is no direct contact between them. In other words, the respective pads distance from each other on the circuit board are disposed on the circuit board to be free of direct contact. In other words, the respective pads do not contact or about each other, but may be electrically connected by appropriate devices e.g., by the first bridge in terms of the electric connection between the first pad of the second type and the second pad of the second type.

### BRIEF DESCRIPTION OF THE DRAWINGS

The foregoing summary as well as the following detailed description of preferred embodiments will be better understood when read in conjunction with the appended drawings. For the purpose of illustrating the present disclosure, reference is made to the drawings. However, the scope of the disclosure is not limited to the specific embodiments disclosed in the drawings.

In the drawings:

FIG. 1 is a side view of a blood pump according to the present disclosure,

FIG. 2 is a front view of the blood pump of FIG. 1,

FIG. 3 is a cross section along the line A-A shown in FIG. 2,

FIG. 4 is a cross section along the line B-B shown in FIG. 3,

FIG. 5 is a perspective view of a circuit board according to the present disclosure,

FIG. 6 is a top view of the circuit board of FIG. 5,

FIG. 7 is a side view of the circuit board of FIG. 5,

FIG. 8 is an enlarged view of the detail X of FIG. 7,

FIG. 9 is a back view of the circuit board of FIG. 5 with further elements indicated,

FIG. 10 is an exploded view of the circuit board of FIG. 5, and

FIG. 11 is an example of the connections between a stator and the circuit board of FIG. 5.

### DETAILED DESCRIPTION

In the Figures, like reference numbers denote similar or identical elements. Further, for lucidity reasons not all reference numbers are depicted in every Figure.

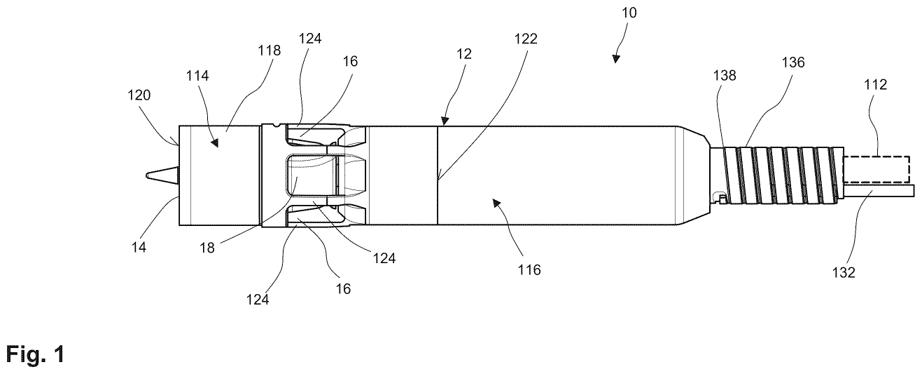

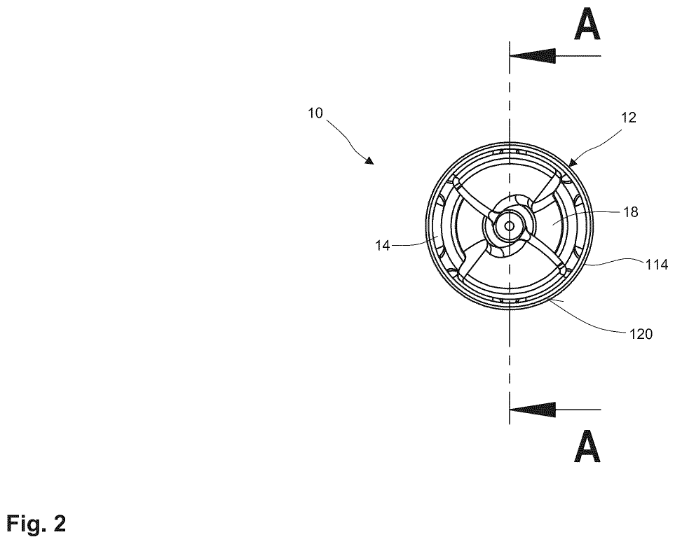

FIG. 1 depicts a side view of a blood pump 10 and FIG. 2 depicts a front view of the blood pump 10. In this exemplary embodiment, the blood pump 10 is an intravascular blood pump for left ventricular support. The blood pump 10 comprises a pumping section in form of a tubular-shaped blood pump housing 12, a shaft 110, a pump element 18, a drive unit 20, a purge fluid line 112 and a cable assembly 132. The purge fluid line 112 is only schematically indicated in FIG. 1, but may be configured as shown in US 2021/0339003 A1, which is hereby incorporated by reference in its entirety. In this exemplary embodiment, the shaft 110 is completely made of aluminum toughened zirconia.

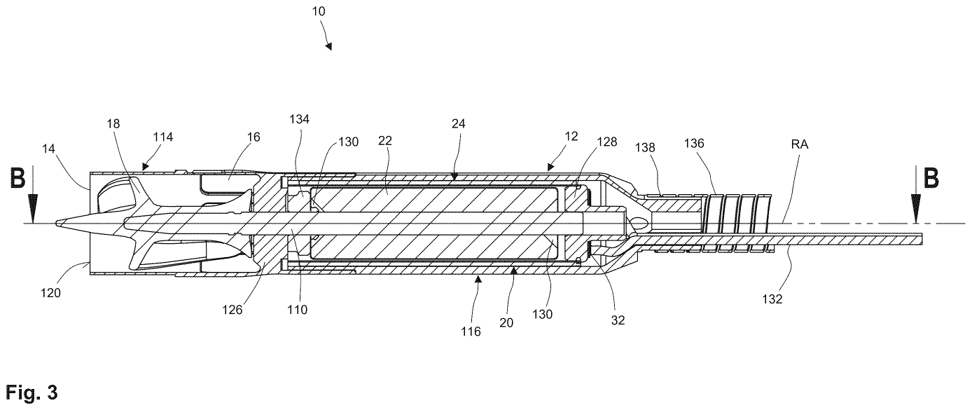

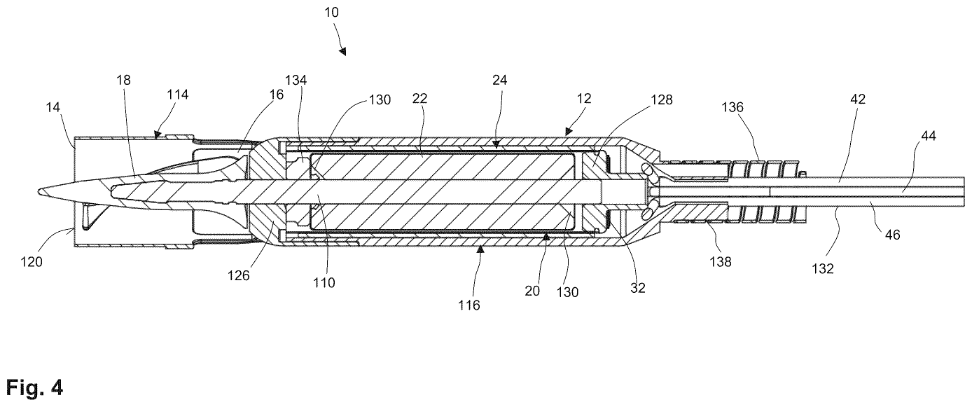

In this exemplary embodiment, the blood pump housing 12 is a multi-part housing and comprises a first housing part 114 and a second housing part 116. The first housing part 114 comprises an outer casing 118, a distal end 120 and a proximal end 122, see also FIGS. 3 and 4. A first blood flow opening 14 is provided at the distal end 120 of the first housing part 114. In addition, at least one second blood flow opening 16 is provided between the distal end 120 and the proximal end 122 of the first housing part 114. In this exemplary embodiment, the first housing part 114 comprises six second blood flow openings 16 which are provided equally spaced around the circumference of the first housing part 114 at the outer casing 118 and which form radial openings of the outer casing 118. The second blood flow openings 16 are separated by struts 124, in this exemplary embodiment by a total of six struts 124.

As shown in the detailed view of FIGS. 3 and 4 (with the purge fluid line 112 not being shown) the end of the blood pump housing 12 opposite to the distal end 120 of the first housing part 114 and the first blood flow opening 14 comprises a tubular portion having a threaded portion 138 on its outer peripheral surface which virtually corresponds to an external thread. The threaded portion 138 is configured to threadedly engage a helical member in form of a Nitinol coil 136. In particular, the Nitinol coil 138 is threaded onto the threaded portion 138 in order to attach e.g., a catheter (not shown) to the pump housing 12. The purge fluid line 112 and the cable assembly 132 may be disposed within the catheter.

The pump element 18 is non-rotatably supported on the shaft 110, so that it is partially disposed within the first housing part 114. In this embodiment, the pump element 18 is an impeller having blades on its outer circumferential surface. The impeller 18 rotates together with the shaft 110 about a central axis of rotation RA, so that blood is conveyed between the first blood flow opening 14 and the plurality of second blood flow openings 16, see also FIGS. 3 and 4. As the blood pump 10 in the shown embodiment is intended for left ventricular support, a blood flow is caused from the first blood flow opening 14 to the plurality of second blood flow openings 16 i.e., the first blood flow opening 14 is a blood flow inlet and each of the second blood flow openings 16 is a blood flow outlet.

As can be seen in the cross section of FIG. 3 and FIG. 4, the shaft 110 is supported at two spaced apart points, namely at a shaft supporting portion 126 in close proximity to the impeller 18 and at a bearing washer 128 disposed axially behind the drive unit 20 in the second housing part 116 when viewed from the distal end 120 of the first housing part 114 to the proximal end 122 of the first housing part 114. Thus, the drive unit 20 is sandwiched between the shaft supporting portion 126 and the bearing washer 128. The bearing washer 128 may be a sliding bearing.

Rotation of the shaft 110 is caused by the drive unit 20. As shown in FIGS. 3 and 4, the drive unit 20 comprises a magnet 22 as a rotor and a stator 24. The magnet 22 is non-rotatably supported on the shaft 110 and is disposed radially within the stator 24. The stator 24 is housed within the blood pump housing 12, in particular within the second housing part 116. In this embodiment, the stator 24 is a multi-layer stator coil, preferably with two layers of windings embedded in a suitable synthetic material or synthetic resin as will be described in more detail below.

The magnet 22 comprises recessed portions 130 on both axial ends which are recessed towards the other one of the axial ends. During assembly of the blood pump 10, the recessed portions 130 are filed with an adhesive. Energizing the stator 24 via the cable assembly 132 causes an alternating magnetic field which induces a force on the magnet 22 so that it rotates together with the shaft 110 about the central axis of rotation RA and, hence, with the impeller 18 to cause the blood flow as described above. Energization of the stator 24 is per se known e.g., from WO 2023/018793 A1 which is hereby incorporated by reference in its entirety.

Furthermore, a washer 134 is supported on the shaft 110 between the shaft supporting portion 126 and the magnet 22. The washer 134 is intended to correctly position the magnet 22 on the shaft 110. The washer 134 may be a thrust washer.

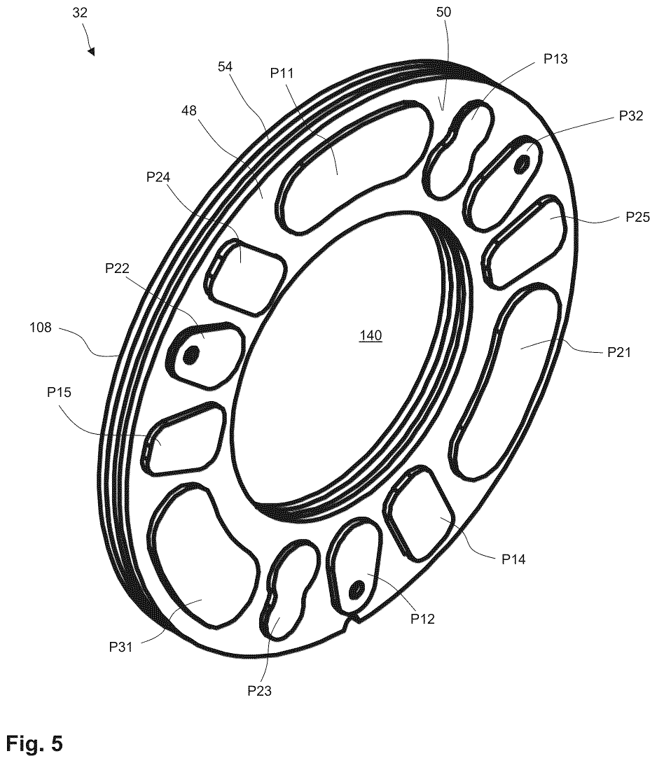

To energize the stator 24 and to generate the alternating magnetic field, the stator 24 is connected to the cable assembly 132 via a circuit board 32. In this exemplary embodiment, the circuit board 32 is a multi-layer printed circuit board 32 having an annular shape or ring shape respectively, see FIGS. 5 to 10. The circuit board 32 has an outer diameter of 2 mm to 4 mm, preferably of 3 mm and comprises a central opening 140 having a diameter of 1 mm to 2 mm, preferably of 1.6 mm. The circuit board 32 is configured to be supported on the bearing washer 128 in that a portion of the bearing washer 128 extends through the central opening 140, see FIG. 3 and FIG. 4.

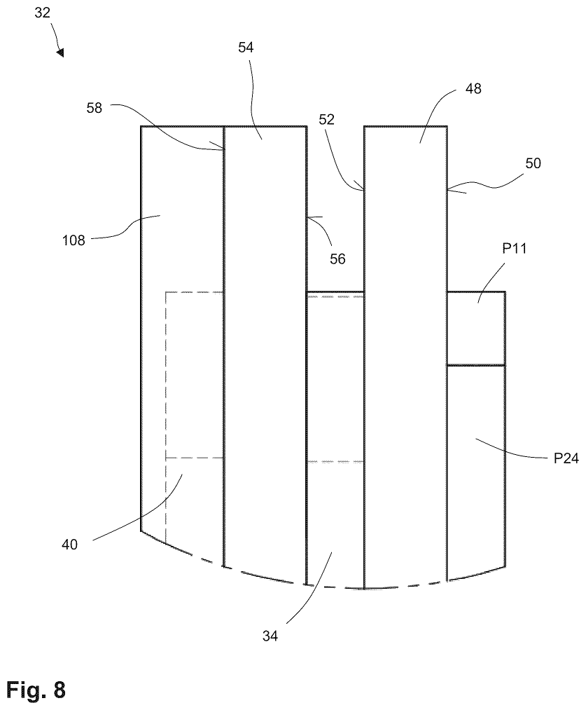

The circuit board 32 comprises a first layer 48, a second layer 54 and an adhesive foil 108, with the second layer 54 being disposed between the first layer 48 and the adhesive foil 108, see e.g., FIG. 8. The first layer 48 and the second layer 54 each have a thickness in the axial direction of 0.02 mm to 0.07 mm, preferably of 0.05 mm. The first layer 48 and the second layer 54 are made of a suitable plastic material e.g., of Polyimide. The adhesive foil 108 has a thickness in the axial direction of 0.02 mm to 0.07 mm, preferably of 0.05 mm. The adhesive foil 108 is configured as a double-side adhesive foil so that it can be attached to the second layer 54 and to the bearing washer 128. The circuit board 32 has a thickness in the axial direction of 0.1 mm to 0.3 mm, preferably of 0.22 mm.

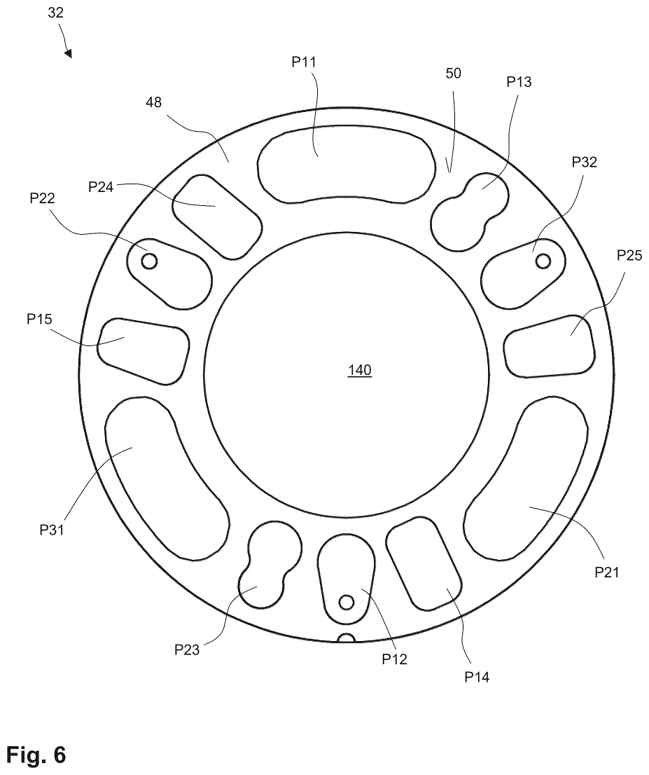

The first layer 48 has a first side 50 facing towards the threaded portion 138 and second side 52 opposite of the first side 50. The second layer 54 has a first side 56 facing the second side 52 of the first layer 48 and a second side 58 facing the adhesive foil 108. A plurality of pads P11 to P25 are disposed on the first side 50 of the first layer 48. As shown in FIGS. 5 and 6, the plurality of pads P11 to P25 are disposed in an annular shape or ring shape on the first side 50 of the first layer 48. The plurality of pads P11 to P25 are composed of gold plated copper. The plurality of pads P11 to P25 each have a thickness in the axial direction of 0.015 mm to 0.055 mm, preferably of 0.035 mm.

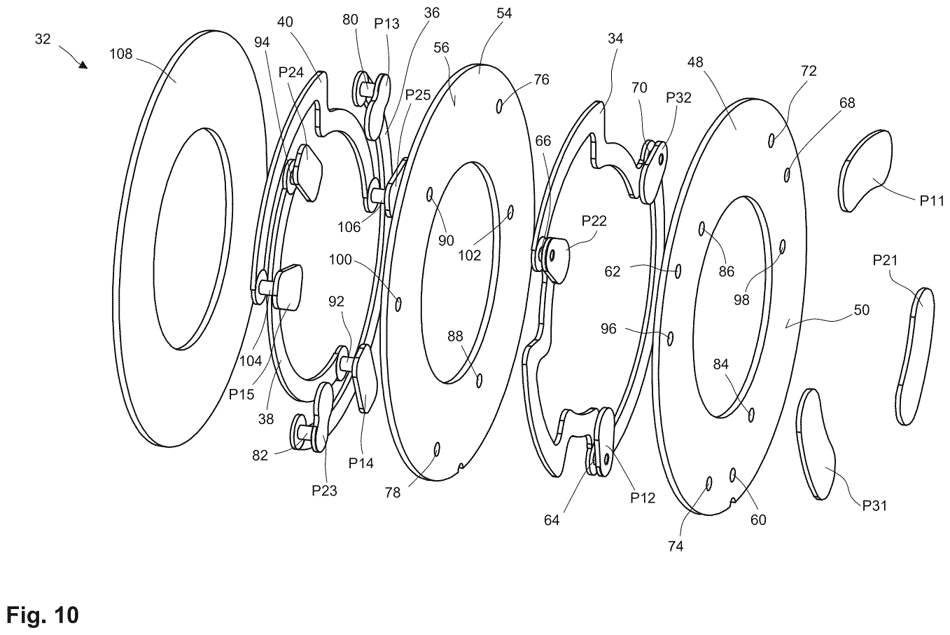

Furthermore, the first layer 48 comprises a first opening 60, a second opening 62, a third opening 68, a fourth opening 72, a fifth opening 74, an eighth opening 84, a ninth opening 86, a twelfth opening 96 and a thirteenth opening 98 which all fully extend through the first layer 48, see FIG. 10. The second layer 54 comprises a sixth opening 76, a seventh opening 78, a tenth opening 88, an eleventh opening 90, a fourteenth opening 100 and a fifteenth opening 102 which all fully extend through the second layer 54, see FIG. 10.

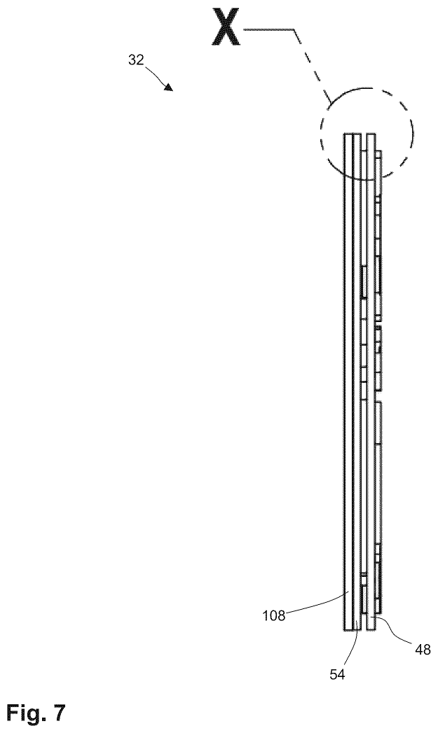

A first bridge 34 is disposed on the second side 52 of the first layer 48 and on the first side 56 of the second layer 54 respectively. Hence, the first bridge 34 is sandwiched between the first layer 48 and the second layer 54, see also FIGS. 7 and 8. The first bridge 34 is made of gold plated copper and has a thickness in the axial direction of 0.015 mm to 0.055 mm, preferably of 0.035 mm.

A second bridge 36 is disposed on the second side 58 of the second layer 54. Hence, the second bridge 36 is sandwiched between the second layer 54 and the adhesive foil 108, see also FIGS. 7 and 8. The second bridge 36 is made of gold plated copper and has a thickness in the axial direction of 0.015 mm to 0.055 mm, preferably of 0.035 mm.

A third bridge 38 is disposed on the second side 58 of the second layer 54. Hence, the third bridge 38 is sandwiched between the second layer 54 and the adhesive foil 108, see also FIGS. 7 and 8. The third bridge 38 is made of gold plated copper and has a thickness in the axial direction of 0.015 mm to 0.055 mm, preferably of 0.035 mm.

A fourth bridge 40 is disposed on the second side 58 of the second layer 54. Hence, the fourth bridge 40 is sandwiched between the second layer 54 and the adhesive foil 108, see also FIGS. 7 and 8. The fourth bridge 40 is made of gold plated copper and has a thickness in the axial direction of 0.015 mm to 0.055 mm, preferably of 0.035 mm.

As can be seen from FIG. 8, the second bridge 36, the third bridge 38 and the fourth bridge 40 are embedded within the adhesive foil 108, whereas the first bridge 34 is sandwiched between the first layer 48 and the second layer 54. Further, the second layer 54 electrically isolates the first bridge 36 from the second to fourth bridges 36 to 40.

The plurality of pads P11 to P25 comprises a first pad of a first type P11, a second pad of the first type P21, a third pad of the first type P31, a first pad of a second type P12, a second pad of the second type P22, a third pad of the second type P32, a first pad of a third type P13, a second pad of the third type P23, a first pad of a fourth type P14, a second pad of the fourth type P24, a first pad of a fifth type P15, and a second pad of the fifth type P25. As depicted in e.g., FIG. 6, the pads P11 to P25 are arranged on the first side 50 of the first layer 48 so that there is always at least one pad of a different type in between two pads of the same type.

In other words, the first pad of the first type P11, the second pad of the first type P21 and the third pad of the first type P31 are distanced from each other on the circuit board 32 or the first layer 48 respectively. As such, the first pad of the second type P12, the second pad of the second type P22 and the third pad of the second type P32 are distanced from each other on the circuit board 32 or the first layer 48 respectively. The first pad of the third type P13 and the second pad of the third type P32 are distanced from each other on the circuit board 32 or the first layer 48 respectively. The first pad of the fourth type P14 and the second pad of the fourth type P24 are distanced from each other on the circuit board 32 or the first layer 48 respectively. The first pad of the fifth type P15 and the second pad of the fifth type P25 are distanced from each other on the circuit board 32 or the first layer 48 respectively.

Furthermore, the first pad of the second type P12, the second pad of the second type P22 and the third pad of the second type P32 are electrically connected to each other via the first bridge 34. In particular, a first connector 64 extends through the first opening 60 and connects the first pad of the second type P12 with the first bridge 34. A second connector 66 extends through the second opening 62 and connects the second pad of the second type P22 with the first bridge 34. A third connector 70 extends through the third opening 68 and connects the third pad of the second type P32 with the first bridge 34.

The first pad of the third type P13 and the second pad of the third type P23 are electrically connected to each other via the second bridge 36. In particular, a fourth connector 80 extends through the fourth opening 72 and the sixth opening 76 and connects the first pad of the first type P13 to the second bridge 36. A fifth connector 82 extends through the fifth opening 74 and the seventh opening 78 and connects the second pad of the third type P23 to the second bridge 36.

The first pad of the fourth type P14 and the second pad of the fourth type P24 are electrically connected to each other via the third bridge 38. In particular, a sixth connector 92 extends through the eighth opening 84 and the tenth opening 88 and connects the first pad of the fourth type P14 to the third bridge 38. A seventh connector 94 extends through the ninth opening 86 and the eleventh opening 90 and connects the second pad of the fourth type P24 to the third bridge 38.

The first pad of the fifth type P15 and the second pad of the fifth type P25 are electrically connected to each other via the fourth bridge 40. In particular, an eighth connector 104 extends through the twelfth opening 96 and the fourteenth opening 100 and connects the first pad of the fifth type P15 to the fourth bridge 40. A ninth connector 106 extends through the thirteenth opening 98 and the fifteenth opening 102 and connects the second pad of the fifth type P25 to the fourth bridge 40.

The first to ninth connector are bolt-shaped and are made of copper and may be gold plated. The first to ninth connector have a diameter 0.08 mm to 0.12 mm, preferably of 0.1 mm.

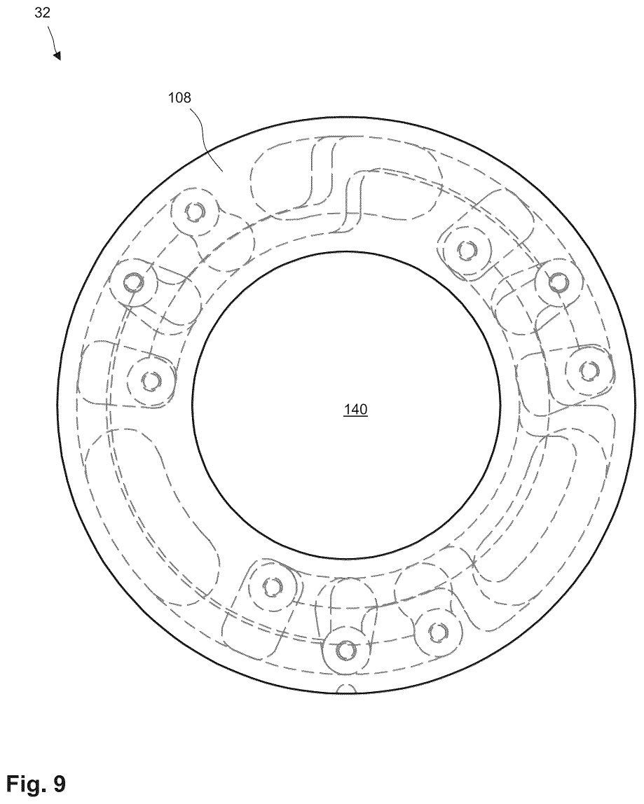

The overall configuration of the pads P11 to P25 and the first to fourth bridge 34 to 40 can be seen in FIG. 9. There, the pads P11 to P25 and the bridges 34 to 40 are denoted by light dashed lines. As can be further derived from FIG. 10, the bridges 34 to 40 are arranged on the circuit board 32 so that there is no electrical connection between them. Accordingly, the pads of the first type P11 to P31, the pads of the second type P12 to P32, the pads of the third type P13 and P23, the pads of the fourth type P14 and P24 and the pads of the fifth type P15 and P25 are arranged so that there is no electrical connection between pads of a different type via the circuit board 32.

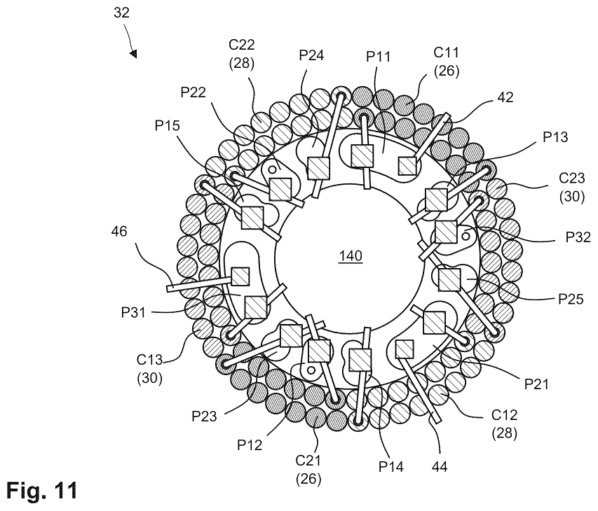

As exemplarily shown in FIG. 11, a first cable 42 of the cable assembly 132 is attached to the first pad of the first type P11, a second cable 44 of the cable assembly 132 is attached to the second pad of the first type P21 and a third cable 46 of the cable assembly 132 is attached to the third pad of the first type P31. The first cable 42, the second cable 44 and the third cable 46 are attached to the respective pad of the first type P11 to P31 via a suitable procedure e.g., by laser soldering or by ultrasonic soldering.

In this exemplary embodiment, the stator 24 comprises a first coil assembly 26, a second coil assembly 28 and a third coil assembly 30. Thus, the drive unit 20 is a three-phase two-pole electric motor. As shown in FIG. 11, the first coil assembly 26 comprises a first coil of a first type C11 and a second coil of the first type C21 being disposed on opposite sides of the stator 24. The first coil of the first type C11 is a multi-layer coil with a plurality of windings. The first coil of the first type C11 is connected to the first pad of the first type P11 via a wire and to the first pad of the third type P13 via a wire. The second coil of the first type C21 is a multi-layer coil with a plurality of windings. The second coil of the first type C21 is connected to the second pad of the third type P23 via a wire and to the first pad of the second type P12 via a wire.

The second coil assembly 28 comprises a first coil of a second type C12 and a second coil of the second type C22 being disposed on opposite sides of the stator 24. The first coil of the second type C12 is a multi-layer coil with a plurality of windings. The first coil of the second type C12 is connected to the second pad of the first type P12 via a wire and to the first pad of the fourth type P14 via a wire. The second coil of the second type C22 is a multi-layer coil with a plurality of windings. The second coil of the second type C22 is connected to the second pad of the fourth type P24 via a wire and to the second pad of the second type P22 via a wire.

The third coil assembly 30 comprises a first coil of a third type C13 and a second coil of the third type C23 being disposed on opposite sides of the stator 24. The first coil of the third type C13 is a multi-layer coil with a plurality of windings. The first coil of the third type C13 is connected to the third pad of the first type P13 via a wire and to the first pad of the fifth type P15 via a wire. The second coil of the third type C23 is a multi-layer coil with a plurality of windings. The second coil of the third type C23 is connected to the second pad of the fifth type P25 via a wire and to the third pad of the second type P32 via a wire.

The wires of the coil assemblies C11 to C23 are attached to the respective pads via a suitable procedure e.g., by laser soldering or by ultrasonic soldering. Accordingly, the coils of the respective coil assemblies 26, 28 and 30 are configured in series via the respective pads and the coil assemblies are configured in a star or wye circuit configuration via the pads of the second type P12 to P32.

Electrical signals applied to the first coil assembly 26 via the first cable 42, to the second coil assembly 28 via the second cable 44 and to the third coil assembly 30 via the third cable 46 thus cause the magnet 22 to rotate together with the impeller 18 so that the intended blood flow is generated.

### EXEMPLARY IMPLEMENTATIONS

As already described, the technology described herein may be implemented in various ways. In that regard, the foregoing disclosure is intended to include, but not be limited to, the systems, methods, and combinations and sub-combinations thereof that are set forth in the following exemplary implementations. Preferred embodiments are described in the following paragraphs:
A1 Blood pump comprising: a pumping section having a first blood flow opening and a second blood flow opening; a pump element being at least partially disposed within the pumping section; a drive unit having a rotor and a stator.

A2 Blood pump according to paragraph A1, wherein the stator comprises a first coil assembly and/or a second coil assembly.
A3 Blood pump according to paragraph A2, wherein the blood pump further comprises a circuit board having a first pad of a first type, a second pad of the first type, a first pad of a second type and a second pad of the second type.
A4 Blood pump according to paragraph A3, wherein the first pad of the second type and the second pad of the second type are distanced from each other on the circuit board.
A5 Blood pump according to paragraph A4, wherein the first pad of the second type is electrically connected to the second pad of the second type by a first bridge.
A6 Blood pump according to paragraph A5, wherein the first coil assembly is connected to the first pad of the first type and to the first pad of the second type.
A7 Blood pump according to paragraph A6, wherein the second coil assembly is connected to the second pad of the first type and to the second pad of the second type.
A8 Blood pump according to any one of the preceding paragraphs A3 to A7, the blood pump further comprising a first cable being attached to the first pad of the first type, and/or a second cable being attached to the second pad of the first type.
A9 Blood pump according to paragraph A8, wherein the first cable and the second cable are configured to apply electrical signals to the first coil assembly and the second coil assembly to rotate the rotor and to drive the pump element so that a blood flow between the first blood flow opening and the second blood flow opening is generated.
A10 Blood pump according to any one of the preceding paragraphs A2 to A9, wherein the first coil assembly comprises a first coil of a first type and a second coil of the first type.
A11 Blood pump according to any one of the preceding paragraphs A1 to A10, wherein the circuit board comprises a first pad of a third type and a second pad of the third type.
A12 Blood pump according to paragraph A11, wherein the first pad of the third type and the second pad of the third type are distanced from each other on the circuit board.
A13 Blood pump according to paragraph A11 or A12, wherein the first pad of the third type is electrically connected to the second pad of the third type by a second bridge.
A14 Blood pump according to any one of the preceding paragraphs A11 to A13, wherein the first coil of the first type is connected to the first pad of the first type to establish the connection between the first coil assembly and the first pad of the first type.
A15 Blood pump according to any one of the preceding paragraphs A11 to A14, wherein the second coil of the first type is connected to the first pad of the second type to establish the connection between the first coil assembly and the first pad of the second type.
A16 Blood pump according to any one of the preceding paragraphs A11 to A15, wherein the first coil of the first type is connected to the first pad of the third type, and/or wherein the second coil of the first type is connected to the second pad of the third type.
A17 Blood pump according to any one of the preceding paragraphs A1 to A16, wherein he second coil assembly comprises a first coil of a second type and a second coil of the second type.
A18 Blood pump according to any one of the preceding paragraphs A1 to A17, wherein the circuit board comprises a first pad of a fourth type and a second pad of the fourth type.
A19 Blood pump according to paragraph A18, wherein the first pad of the fourth type and the second pad of the fourth type are distanced from each other on the circuit board.
A20 Blood pump according to paragraph A18 or A19, wherein the first pad of the fourth type is electrically connected to the second pad of the fourth type by a third bridge.
A21 Blood pump according to any one of the preceding paragraphs A17 to A20, wherein the first coil of the second type is connected to the second pad of the first type to establish the connection between the second coil assembly and the second pad of the first type.
A22 Blood pump according to any one of the preceding paragraphs A17 to A21, wherein the second coil of the second type is connected to the second pad of the second type to establish the connection between the second coil assembly and the second pad of the second type.
A23 Blood pump according to any one of the preceding paragraphs A18 to A22, wherein the first coil of the second type is connected to the first pad of the fourth type and/or wherein the second coil of the second type is connected to the second pad of the fourth type.
A24 Blood pump according to any one of the preceding paragraphs A1 to A23, wherein the stator comprises a third coil assembly.
A25 Blood pump according to paragraph A24, wherein the circuit board comprises a third pad of the first type and a third pad of the second type.
A26 Blood pump according to paragraph A25, wherein the first pad of the second type and the second pad of the second type and the third pad of the second type are distanced from each other on the circuit board.
A27 Blood pump according to paragraph A25 or A26, wherein the third pad of the second type is electrically connected to the first pad of the second type and the second pad of the second type by the first bridge.
A28 Blood pump according to any one of the preceding paragraphs A23 to A27, wherein the third coil assembly is connected to the third pad of the first type and to the third pad of the second type.
A29 Blood pump according to any one of the preceding paragraphs A25 to A28, wherein the blood pump further comprises a third cable being attached to the third pad of the first type.
A30 Blood pump according to paragraph A29, wherein the third cable is configured to apply electrical signals to the third coil assembly.
A31 Blood pump according to any one of the preceding paragraphs A24 to A30, wherein the third coil assembly comprises a first coil of a third type and a second coil of the third type.
A32 Blood pump according to paragraph A31, wherein the circuit board comprises a first pad of a fifth type and a second pad of the fifth type.
A33 Blood pump according to paragraph A32, wherein the first pad of the fifth type and the second pad of the fifth type are distanced from each other on the circuit board.
A34 Blood pump according to paragraph A32 or A33, wherein the first pad of the fifth type is electrically connected to the second pad of the fifth type by a fourth bridge.
A35 Blood pump according to any one of the preceding paragraphs A32 to A34, wherein the first coil of the third type is connected to the third pad of the first type to establish the connection between the third coil assembly and the third pad of the first type.
A36 Blood pump according to any one of the preceding paragraphs A32 to A35, wherein the second coil of the third type is connected to the third pad of the second type to establish the connection between the third coil assembly and the third pad of the second type.
A37 Blood pump according to any one of the preceding paragraphs A32 to A36, wherein the first coil of the third type is connected to the first pad of the fifth type, and/or wherein the second coil of the third type is connected to the second pad of the fifth type.
A38 Blood pump according to any one of the preceding paragraphs A3 to A37, wherein the circuit board comprises a first layer having a first side and a second side.
A39 Blood pump according to paragraph A38, wherein the first pad of the first type and/or the second pad of the first type and/or the first pad of the second type and/or the second pad of the second type are disposed on the first side of the first layer, and/or wherein the first bridge is disposed on the second side of the first layer.
A40 Blood pump according to paragraph A38 or A39, wherein the first layer comprises a first opening, wherein the circuit board comprises a first connector wherein the first connector extends through the first opening, wherein the first bridge is connected to the first connector, and wherein the first pad of the second type is connected to the first connector.
A41 Blood pump according to any one of the preceding paragraphs A38 to A40, wherein the first layer comprises a second opening, wherein the circuit board comprises a second connector, wherein the second connector extends through the second opening, wherein the first bridge is connected to the second connector, and wherein the second pad of the second type is connected to the second connector.
A42 Blood pump according to any one of the preceding paragraphs A38 to A41, wherein the third pad of the first type and the third pad of the second type are disposed on the first side (50) of the first layer.
A43 Blood pump according to any one of the preceding paragraphs A38 to A42, wherein the first layer comprises a third opening and a third connector extending through the third opening, wherein the first bridge is connected to the third connector, and wherein the third pad of the second type is connected to the third connector.
A44 Blood pump according to any one of the preceding paragraphs A3 to A43, wherein the circuit board comprises a second layer having a first side and a second side.
A45 Blood pump according to paragraph A44, wherein the first bridge is disposed on the first side of the second layer.
A46 Blood pump according to any one of the preceding claims A38 to A45, wherein the first pad of the third type and/or the second pad of the third type are disposed on the first side of the first layer, and/or the first pad of the fourth type and/or the second pad of the fourth type are disposed on the first side of the first layer, and/or wherein the first pad of the fifth type and/or the second pad of the fifth type are disposed on the first side of the first layer.
A47 Blood pump according to any one of the preceding paragraphs A44 to A46, wherein the second bridge is disposed on the second side of the second layer.
A48 Blood pump according to any one of the preceding paragraphs A44 to A47, wherein the first layer comprises a fourth opening and the second layer comprises a sixth opening, wherein the circuit board comprises a fourth connector, wherein the fourth connector extends through the fourth opening and the sixth opening, wherein the second bridge is connected to the fourth connector, and wherein the first pad of the third type is connected to the fourth connector.
A49 Blood pump according to any one of the preceding paragraphs A44 to A48, wherein the first layer comprises a fifth opening and the second layer comprises a seventh opening, wherein the circuit board comprises a fifth connector, wherein the fifth connector extends through the fifth opening and the seventh opening, wherein the second bridge the fifth connector, and wherein the second pad of the third type is connected to the fifth connector.
A50 Blood pump according to any one of the preceding paragraphs A44 to A49, wherein the third bridge is disposed on the second side of the second layer.
A51 Blood pump according to any one of the preceding paragraphs A44 to A50, wherein the first layer comprises an eighth opening and the second layer comprises a tenth opening, wherein the circuit board comprises a sixth connector, wherein the sixth connector extends through the eighth opening and the tenth opening, wherein the third bridge is connected to the sixth connector, and wherein the first pad of the fourth type is connected to the sixth connector.
A52 Blood pump according to any one of the preceding paragraphs A44 to A51, wherein the first layer comprises an a ninth opening and the second layer comprises an eleventh opening, wherein the circuit board comprises a seventh connector, wherein the seventh connector extends through the ninth opening and the eleventh opening, wherein the third bridge is connected to the seventh connector, and wherein the second pad of the fourth type is connected to the seventh connector.
A53 Blood pump according to any one of the preceding paragraphs A44 to A52, wherein the fourth bridge is disposed on the second side of the second layer.
A54 Blood pump according to any one of the preceding paragraphs A44 to A53, wherein the first layer comprises a twelfth opening and the second layer comprises a fourteenth opening, wherein the circuit board comprises an eighth connector, wherein the eighth connector extends through the twelfth opening and the fourteenth opening, wherein the fourth bridge is connected to the eighth connector, and wherein the first pad of the fifth type is connected to the eighth connector.
A55 Blood pump according to any one of the preceding paragraphs A44 to A54, wherein the first layer comprises a thirteenth opening and the second layer comprises a fifteenth opening, wherein the circuit board comprises a ninth connector, wherein the ninth connector extends through the thirteenth opening and the fifteenth opening, wherein the fourth bridge is connected to the ninth connector, and wherein the second pad of the fifth type is connected to the ninth connector.
A56 Blood pump according to any one of the preceding paragraphs A1 to A55, wherein the circuit board comprises an adhesive foil.
A57 Blood pump according to paragraph A56, wherein the adhesive foil is disposed on an opposite side of the first side of the first layer, preferably on the second side of the second layer.
A58 Blood pump according to any one of the preceding paragraphs A3 to A57, wherein the pads are disposed in an annular pattern on the circuit board.
A59 Blood pump according to paragraph A58, wherein in between two pads of the same type at least one pad of a different type is located.
A60 Blood pump according to any one of the preceding paragraphs A1 to A59, wherein the blood pump is an intravascular blood pump.
A61 Blood pump according to any one of the preceding paragraphs A1 to A60, wherein the pump element is an impeller.
A62 Blood pump according to any one of the preceding paragraphs A1 to A60, wherein the drive unit is a three phase electric motor, preferably three phase two pole electric motor.
A63 Blood pump according to any one of the preceding paragraphs A3 to A62, wherein the circuit board is a printed circuit board.
A64 Blood pump according to any one of the preceding paragraphs A3 to A63, wherein the circuit board has an annular shape.
A65 Blood pump according to any one of the preceding paragraphs A3 to A64, wherein the circuit board has a central opening.
A 66 Blood pump according to paragraph A 65, wherein the central opening has a diameter of 1 mm to 2 mm, preferably of 1.6 mm.
A67 Blood pump according to any one of the preceding paragraphs A3 to A66, wherein the circuit board has an outer diameter of 2 mm to 4 mm, preferably of 3 mm.
A68 Blood pump according to any one of the preceding paragraphs A3 to A67, wherein the circuit board has a thickness in the axial direction of 0.1 mm to 0.3 mm, preferably of 0.22 mm.
A69 Blood pump according to any one of the preceding paragraphs A38 to A68, wherein the first layer has a thickness in the axial direction of 0.02 mm to 0.07 mm, preferably of 0.05 mm.
A70 Blood pump according to any one of the preceding paragraphs A38 to A69, wherein the first layer comprise a polyimide or is composed of a polyimide.
A71 Blood pump according to any one of the preceding paragraphs A44 to A70, wherein the second layer has a thickness in the axial direction of 0.02 mm to 0.07 mm, preferably of 0.05 mm.
A72 Blood pump according to any one of the preceding paragraphs A44 to A71, wherein the second layer comprise a polyimide or is composed of a polyimide.
A73 Blood pump according to any one the preceding paragraphs A3 to A72, wherein at least one or even all of the pads comprise copper or are made of copper.
A74 Blood pump according to any one of the preceding paragraphs A3 to A73, wherein at least one or even all of the pads are gold plated.
A75 Blood pump according to any one of the preceding paragraphs A3 to A74, wherein at least one or even all of the pads have a thickness in the axial direction of 0.015 mm to 0.055 mm, preferably of 0.035 mm.
A76 Blood pump according to any one of the preceding paragraphs A5 to A75, wherein at least one or even all of the bridges comprise copper or are made of copper.
A76 Blood pump according to any one of the preceding paragraphs A5 to A76, wherein at least one or even all of the bridges are gold plated.
A77 Blood pump according to any one of the preceding paragraphs A5 to A77, wherein at least one or even all of the bridges have a thickness in the axial direction of 0.015 mm to 0.055 mm, preferably of 0.035 mm.
A78 Blood pump according to any one of the preceding paragraphs A56 to A77, wherein the adhesive foil has a thickness in the axial direction of 0.02 mm to 0.07 mm, preferably of 0.05 mm.
A79 Blood pump according to any one the preceding paragraphs A40 to A78, wherein at least one or even all of the opening have diameter of 0.05 mm to 0.15 mm, preferably of 0.1 mm.
A80 Blood pump according to any one of the preceding paragraphs A40 to A79, wherein at least one or even all of the connectors comprise copper or are made of copper.
A81 Blood pump according to any one of the preceding paragraphs A40 to A80, wherein at least one or even all of the connectors are gold plated.
A82 Blood pump according to any one of the preceding paragraphs A40 to A81, wherein one or all connectors are bolt-shaped.
A83 Blood pump according to any one of the preceding paragraphs A40 to A82, wherein At least one or even all connectors have a diameter 0.08 mm to 0.12 mm, preferably of 0.1 mm.
A84 Blood pump according to any one of the preceding paragraphs A1 to A83, wherein the blood pump comprises a Nitinol coil.
A85 Blood pump according to paragraph A84, wherein the Nitinol coil is attached to the pumping section.
A86 Blood pump according to paragraph A84 or A85, wherein the pumping section comprises a threaded portion, wherein the Nitinol coil is screwed to the treaded portion.
A87 Blood pump according to any one of the preceding paragraphs A1 to A86, wherein the pumping section is a pump housing.

As utilized herein, the terms “approximately”, “about”, “substantially” and similar terms are intended to have a broad meaning in harmony with the common and accepted usage by those of ordinary skill in the art to which the subject matter of this disclosure pertains. As utilized herein, “proximal” and “distal” are seen relative to the medical staff or physician. Thus, proximal designates something which is relatively close to the physician whereas distal designates something which is relatively far away from the physician when the blood pump is introduced into the patient's body. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure. The terms ,,at least partially” or “partially” as used herein mean both partial and entirely or complete respectively. Terms like “first”, “second” or “third” do not denote a specific order, but are only intended to semantically differentiate between the elements.

### LIST OF REFERENCE SIGNS

10 blood pump
12 pumping section/pump housing
14 first blood flow opening
16 second blood flow opening
18 pump element/impeller
20 drive unit
22 rotor/magnet
24 stator
26 first coil assembly
28 second coil assembly
30 third coil assembly
32 circuit board
34 first bridge
36 second bridge
38 third bridge
40 fourth bridge
42 first cable
44 second cable
46 third cable
48 first layer
50 first side of first layer
52 second side of first layer
54 second layer
56 first side of second layer
58 second side of second layer
60 first opening
62 second opening
64 first connector
66 second connector
68 third opening
70 third connector
72 fourth opening
74 fifth opening
76 sixth opening
80 fourth connector
82 fifth connector
84 eighth opening
86 ninth opening
88 tenth opening
90 eleventh opening
92 sixth connector
94 seventh connector
96 twelfth opening
98 thirteenth opening
100 fourteenth opening
102 fifteenth opening
104 eighth connector
106 ninth connector
108 adhesive foil
110 shaft
112 purge fluid line
114 first housing part
116 second housing part
118 outer casing
120 distal end
122 proximal end
124 strut
126 shaft supporting portion
128 bearing washer
130 recessed portion of magnet
132 cable assembly
134 washer
136 Nitinol coil
138 threaded portion
140 central opening
C11 first coil of first type
C21 second coil of first type
C12 first coil of second type
C22 second coil of second type
C13 first coil of third type
C23 second coil of third type
P11 first pad of first type
P21 second pad of first type
P31 third pad of first type
P12 first pad of second type
P22 second pad of second type
P32 third pad of second type
P13 first pad of third type
P23 second pad of third type
P14 first pad of fourth type
P24 second pad of fourth type
P15 first pad of fifth type
P25 second pad of fifth type
RA axis of rotation

1. A blood pump comprising:
a pumping section having a first blood flow opening and a second blood flow opening;
a pump element being at least partially disposed within the pumping section;
a drive unit having a rotor and a stator, wherein the stator comprises a first coil assembly and a second coil assembly;
a circuit board having a first pad of a first type, a second pad of the first type, a first pad of a second type and a second pad of the second type, wherein the first pad of the second type and the second pad of the second type are distanced from each other on the circuit board, wherein the first pad of the second type is electrically connected to the second pad of the second type by a first bridge, wherein the first coil assembly is connected to the first pad of the first type and to the first pad of the second type, and wherein the second coil assembly is connected to the second pad of the first type and to the second pad of the second type;
a first cable being attached to the first pad of the first type; and
a second cable being attached to the second pad of the first type;
wherein the first cable and the second cable are configured to apply electrical signals to the first coil assembly and the second coil assembly to rotate the rotor and to drive the pump element so that a blood flow between the first blood flow opening and the second blood flow opening is generated.

2. The blood pump according to claim 1, wherein
the first coil assembly comprises a first coil of a first type and a second coil of the first type,
the circuit board comprises a first pad of a third type and a second pad of the third type,
the first pad of the third type and the second pad of the third type are distanced from each other on the circuit board,
the first pad of the third type is electrically connected to the second pad of the third type by a second bridge,
the first coil of the first type is connected to the first pad of the first type to establish a connection between the first coil assembly and the first pad of the first type,
the second coil of the first type is connected to the first pad of the second type to establish a connection between the first coil assembly and the first pad of the second type,
the first coil of the first type is connected to the first pad of the third type, and
the second coil of the first type is connected to the second pad of the third type.

3. The blood pump according to claim 1, wherein
the second coil assembly comprises a first coil of a second type and a second coil of the second type,
the circuit board comprises a first pad of a fourth type and a second pad of the fourth type,
the first pad of the fourth type and the second pad of the fourth type are distanced from each other on the circuit board,
the first pad of the fourth type is electrically connected to the second pad of the fourth type by a third bridge,
the first coil of the second type is connected to the second pad of the first type to establish a connection between the second coil assembly and the second pad of the first type,
the second coil of the second type is connected to the second pad of the second type to establish a connection between the second coil assembly and the second pad of the second type,
the first coil of the second type is connected to the first pad of the fourth type, and
the second coil of the second type is connected to the second pad of the fourth type.

4. The blood pump according to claim 3, wherein
the stator comprises a third coil assembly, and the circuit board comprises a third pad of the first type and a third pad of the second type,
the first pad of the second type and the second pad of the second type and the third pad of the second type are distanced from each other on the circuit board,
the third pad of the second type is electrically connected to the first pad of the second type and the second pad of the second type by the first bridge, the third coil assembly is connected to the third pad of the first type and to the third pad of the second type, and
the blood pump further comprises a third cable being attached to the third pad of the first type, wherein the third cable is configured to apply electrical signals to the third coil assembly.

5. The blood pump according to claim 4, wherein
the third coil assembly comprises a first coil of a third type and a second coil of the third type,
the circuit board comprises a first pad of a fifth type and a second pad of the fifth type,
the first pad of the fifth type and the second pad of the fifth type are distanced from each other on the circuit board,
the first pad of the fifth type is electrically connected to the second pad of the fifth type by a fourth bridge,
the first coil of the third type is connected to the third pad of the first type to establish a connection between the third coil assembly and the third pad of the first type,
the second coil of the third type is connected to the third pad of the second type to establish a connection between the third coil assembly and the third pad of the second type, the first coil of the third type is connected to the first pad of the fifth type, and
the second coil of the third type is connected to the second pad of the fifth type.

6. The blood pump according to claim 5, wherein
the circuit board comprises a first layer having a first side and a second side,
the first pad of the first type and the second pad of the first type and the first pad of the second type and the second pad of the second type are disposed on the first side of the first layer, and
the first bridge is disposed on the second side of the first layer.

7. The blood pump according to claim 6, wherein
the first layer comprises a first opening and a second opening,
the circuit board comprises a first connector and a second connector,
the first connector extends through the first opening,
the second connector extends through the second opening,
the first bridge is connected to the first connector and to the second connector,
the first pad of the second type is connected to the first connector, and
the second pad of the second type is connected to the second connector.

8. The blood pump according to claim wherein
the third pad of the first type and the third pad of the second type are disposed on the first side of the first layer,
the first layer comprises a third opening and the circuit board comprises a third connector extending through the third opening,
the first bridge is connected to the third connector, and the third pad of the second type is connected to the third connector.

9. The blood pump according to claim 6, wherein
the circuit board comprises a second layer having a first side and a second side, and
the first bridge is disposed on the first side of the second layer.

10. The blood pump according to claim 6, wherein
the first pad of the third type and the second pad of the third type are disposed on the first side of the first layer, and/or
the first pad of the fourth type and the second pad of the fourth type are disposed on the first side of the first layer, and/or
the first pad of the fifth type and the second pad of the fifth type are disposed on the first side of the first layer.

11. The blood pump according to claim 9, wherein
the second bridge is disposed on the second side of the second layer,
the first layer comprises a fourth opening and a fifth opening and the second layer comprises a sixth opening and a seventh opening,
the circuit board comprises a fourth connector and a fifth connector,
the fourth connector extends through the fourth opening and the sixth opening and the fifth connector extends through the fifth opening and the seventh opening,
the second bridge is connected to the fourth connector and the fifth connector, and
the first pad of the third type is connected to the fourth connector and the second pad of the third type is connected to the fifth connector.

12. The blood pump according to claim 9, wherein
the third bridge is disposed on the second side of the second layer,
the first layer comprises an eighth opening and a ninth opening and the second layer comprises a tenth opening and an eleventh opening,
the circuit board comprises a sixth connector and a seventh connector,
the sixth connector extends through the eighth opening and the tenth opening and the seventh connector extends through the ninth opening and the eleventh opening,
the third bridge is connected to the sixth connector and the seventh connector, and
the first pad of the fourth type is connected to the sixth connector and the second pad of the fourth type is connected to the seventh connector.

13. The blood pump according to claim 9, wherein
the fourth bridge is disposed on the second side of the second layer,
the first layer comprises a twelfth opening and a thirteenth opening and the second layer comprises a fourteenth opening and a fifteenth opening,
the circuit board comprises an eighth connector and a ninth connector,
the eighth connector extends through the twelfth opening and the fourteenth opening and the ninth connector extends through the thirteenth opening and the fifteenth opening,
the fourth bridge is connected to the eighth connector and the ninth connector, and
the first pad of the fifth type is connected to the eighth connector and the second pad of the fifth type is connected to the ninth connector.

14. The blood pump according to claim, wherein the circuit board comprises an adhesive foil on an opposite side of the first side of the first layer

15. The blood pump according to claim 1, wherein
the pads are disposed in an annular pattern on the circuit board, and
in between two pads, of a same type at least one pad of a different type is located.
